(19) United States
(12) Patent Application Publication
(10) Pub. No.: US 2016/0200642 A1
(43) Pub. Date: Jul. 14, 2016

(54) ZEOLITE CATALYSTS FOR THE CONVERSION OF ALKYL HALIDES TO OLEFINS

(71) Applicants: Ashim Kumar GHOSH, Sugar Land, TX (US); Mike MIER, Sugar Land, TX (US); SABIC GLOBAL TECHNOLOGIES B.V., Bergen op Zoom (NL)

(72) Inventors: Ashim Kumar Ghosh, Houston, TX (US); Mike Mier, Waller, TX (US)

(73) Assignee: Sabic Global Technologies B.V., Bergen op Zoom (NL)

(21) Appl. No.: 14/899,003

(22) PCT Filed: Jun. 29, 2015
(86) PCT No.: PCT/US2015/038332

§ 371 Date: Dec. 16, 2015

Related U.S. Application Data
(60) Provisional application No. 62/023,516, filed on Jul. 11, 2014.

(51) Int. Cl. C07C 1/30; C01B 39/38; C08F 10/00; B01J 29/40

(52) U.S. Cl. CPC C07C 1/30; B01J 29/40; C01B 39/38; C08F 10/00; C07C 2529/40

(57) ABSTRACT

Disclosed is a method for converting an alkyl halide to an olefin. The method can include contacting a zeolite catalyst comprising HZSM-5 having a silica to alumina (SAR) ratio of at least 30 with a feed that includes an alkyl halide under reaction conditions sufficient to produce an olefin hydrocarbon product comprising C₂ to C₄ olefins, wherein the selectivity of the C₂ to C₄ olefins is at least 85% at 20% alkyl halide conversion.

### CROSS REFERENCE TO RELATED APPLICATIONS

This application claims benefit to U.S. Provisional Patent Application No. 62/023,516, titled “ZEOLITE CATALYSTS FOR THE CONVERSION OF ALKYL HALIDES TO OLEFINS”, filed Jul. 11, 2014. The entire contents of the referenced application is incorporated herein by reference.

### BACKGROUND OF THE INVENTION

A. Field of the Invention

The invention generally concerns the use of ZSM-5 zeolites as catalysts in the production of C₂-C₄ olefins from alkyl halides. In particular, the ZSM-5 zeolite can have a silica to alumina (SiO₂/Al₂O₃) ratio of greater than about 30, a high selectivity for propylene and butylene production, and improved stable catalyst performance over prolonged periods of use.

B. Description of Related Art

Descriptions of units, abbreviation, terminology, etc. used throughout the present invention are summarized in Table 1.

Light olefins such as ethylene and propylene are used by the petrochemical industry to produce a variety of key chemicals that are then used to make numerous downstream products. By way of example, both of these olefins are used to make a multitude of plastic products that are incorporated into many articles and goods of manufacture. FIG. 1 is a chart that provides non-limiting uses of propylene. Currently, the main process used to prepare light olefins is via steam cracking of naphtha. This process, however, requires large amounts of naphtha, which in-turn, is obtained from the distillation of crude oil. While this process is viable, its reliance on crude oil can be a rate-limiting step and can increase the manufacturing costs associated with ethylene and propylene production.

Methane activation to higher hydrocarbons, specifically to light olefins, has been the subject of great interest over many decades. Recently, the conversion of methane to light olefins via a two-step process that includes conversion of methane to methyl halide, particularly to methyl mono-halide, for example, to methyl chloride, followed by conversion of the halide to light olefins has attracted great attention. Typically, zeolite (e.g., ZSM-5) or zeolite type catalysts (e.g., SAPO-34) have been tried for the methyl chloride (or other methyl halide) conversion. However, the selectivity to a desired olefin (e.g., propylene) and the rapid catalyst deactivation for the halide reaction remain the major challenges for commercial success.

One of the most commonly used catalysts in petrochemical industry is ZSM-5 zeolite. It is a medium pore zeolite with pore size about 5.5 Å and is shown to convert methyl halide, particularly methyl chloride or methyl bromide, to C₂-C₄ olefins and aromatics under methyl halide reaction conditions. Whereas molecular sieve SAPO-34, an isostructure of chabazite zeolite having small pore openings (3.8 Å), is shown to convert methyl halide to ethylene and propylene and small amounts of C₄ olefins. Both catalysts, however, are shown to deactivate rapidly during methyl halide conversion due to carbon deposition on the catalysts.

Recently, an attempt has been made to produce propylene from methyl chloride and methyl bromide (see Xu et al. Fluoride-treated HZSM-5 as a highly selective stable catalyst for the production of propylene from methyl halides, Journal of Catalysis, Vol. 295, November 2012, pp. 232-241). The collaborators in Xu et al. treated a ZSM-5 catalyst with fluoride to increase both the propylene selectivity and stability of the catalyst. Notably, however, the collaborators observed that untreated HZSM-5 catalysts showed considerable catalyst deactivation. Such deactivation of the catalyst requires frequent or continuous catalyst regeneration or frequent catalyst change-out resulting in inefficient plant operation or in the use of more catalysts to produce the desired amounts of ethylene and propylene, which in turn increases the manufacturing costs. Still further, the catalytic material has to be re-supplied in shorter time intervals, which oftentimes requires the reaction process to be shut down.

TABLE 1

| Abbreviation | Description |
|---|---|
| Å | Angstrom |
| ° C. | degree Celsius |
| ° C./min | degree Celsius per minute |
| cm³/min | cubic centimeter per min |
| g | Gram |
| g/cm³ | gram per cubic centimeter |
| h | Hour |
| m²/g | meter square per gram |
| mole % | mole percent |
| mmole/g-cat | millimole per gram of catalyst |
| NH₃-TPD | ammonia temperature programmed desorption |
| OD | outer diameter |
| % | Percent |
| psig | pound per square inch gauge |
| WHSV | weight hourly space velocity |
| wt % | weight percent |

### SUMMARY OF THE INVENTION

A discovery has been made that solves the problems associated with low molecular weight olefin production. In particular, the discovery is premised on the use of HZSM-5 catalysts to convert alkyl halides to C₂ to C₄ olefins. The catalysts of the present invention have shown increased selectivity towards propylene and butylene production as well as increased catalyst performance stability during prolonged periods of use. That is, the loss in catalytic activity begins to taper and stabilize during use, thereby allowing for the continued use of the catalysts over longer periods of time. Without wishing to be bound by theory, it is believed that a HZSM-5 zeolite catalyst with a silica to alumina ratio (SAR) of greater than about 30 but less than 1000, preferably 30 to 150 or more preferably 30 to 100 or even more preferably 50 to 100, provides both an increased selectivity for the production of propylene and butylene from alkyl halides while also providing increased stable catalytic performance over prolonged periods of use.

In one aspect of the present invention, there is disclosed a method for converting an alkyl halide to an olefin. The method can include contacting a zeolite catalyst with a feed that can include an alkyl halide under reaction conditions sufficient to produce an olefin hydrocarbon product (e.g., propylene and butylene). The zeolite catalyst can include HZSM-5 having a silica to alumina (SAR) ratio of at least about 30 but less than about 1000, preferably 30 to 150, more preferably 30 to 100, and even more preferably 50 to 100, and the selectivity of propylene and butylene can be at least 60%, at reaction conditions including weight hourly space velocity (WHSV) of the alkyl halide feed greater than 0.5 h⁻¹, preferably 0.5 to 10 h⁻¹, more preferably 0.5 to 5 h⁻¹, most preferably 0.7 to 3.0 h⁻¹, a reaction temperature between 300° C. to 500° C. preferably between 350° C. to 400° C. and at less than 20 psig preferably at less than 5 psig. In more particular instances, the SAR ratio can be equal to or greater than 30, or is 50 to 500 or is 50 to 700, or is 80 to 500, or is 30 to 450, or is 30 to 150, or is 50 to 100. In other instances, the SAR ratio is at least 30 and less than 200 or less than 250, or is greater than 50 and less than 260, or is greater than 80 and less than 250. In still other instances, the SAR ratio is at least 30 to 1000. In particular aspects, the selectivity of propylene and butylene is at least 60%. In certain instances, the selectivity of propylene and butylene is at least about 80%. In certain instances, the selectivity of propylene is 30 to 60% and the selectivity of butylene is 25 to 35%. In certain instances, the C₂-C₄ olefin selectivity is greater than 85%. Further, the selectivity to C₂-C₄ alkane selectivity is less than about 1% under certain alkyl halide reaction conditions. Still further, the selectivity of aromatic compounds produced during the process is less than 1% or less than 0.5% and/or the selectivity of ethylene produced during the process is less than 10% or less than 7% or less than 5%, or less than 4%, 3%, 2%, or 1%. The alkyl halide comprised within the feed can have the following structure: CₙH₍₂ₙ₊₂₎₋ₘXₘ, where n is an integer less than 5, preferably n is less than 3, more preferably n is 1, X is Br, F, I, or Cl, and m is an integer between 1 and 3, preferably m is less than or equal to 2, more preferably m is 1. The feed can include about 10, 15, 20, 40, 50 mole % or more of an alkyl halide such as methyl halide. In particular aspects, the feed can include about 10 to 30 or about 20 mole % of the alkyl halide. Non-limiting examples of methyl halides include methyl chloride, methyl bromide, methyl fluoride, or methyl iodide, or any combination thereof. In particular embodiments, the alkyl halide is methyl chloride or methyl bromide. The method can further include collecting or storing the produced olefin hydrocarbon product along with using the produced olefin hydrocarbon product to produce a petrochemical or a polymer. Also, the zeolite catalyst can include, in addition to or in lieu of ZSM-5, any one of or any combination of ZSM-11, ZSM-23, silicalite, ferrierite, and mordenite. Still further, the zeolite catalyst can be one that has not been treated with metal or a halide. In particular aspects, the zeolite catalyst has not been treated with phosphorus or a halide (e.g., it has not been subjected to fluoride treatment) or ion-exchanged with cations (e.g., Cs⁺, Ca²⁺, etc.) or deposited with metal (e.g., Pt, Pd, etc.).

In particular instances, the loss in activity of the zeolite catalyst begins to taper after 15 hours of use at a temperature of 325 to 375° C., such that its activity remains substantially constant (e.g., the conversion rate of the alkyl halide by the zeolite catalyst does not vary by more than 10%, 5%, 4%, 3%, or 2% between 15 to 20 hours of use under certain reaction conditions of a temperature of 325 to 375° C., WHSV of CH₃Cl 0.7 to 1.1 h¹, and at reactor inlet pressure less than 5 psig). The method can further include collecting or storing the produced olefin hydrocarbon product along with using the produced olefin hydrocarbon product to produce a petrochemical or a polymer. Additionally, the used and deactivated zeolite catalyst can be regenerated (e.g., after 5, 10, 15, 20, 25, or 30 hours of use, the catalyst can be regenerated).

In some instances, the catalyst can convert at least 10 g of CH₃Cl per g of catalyst at WHSV of CH₃Cl of about 3 h⁻¹ and at reactor pressure less than 5 psig maintaining CH₃Cl conversion at least 20% at a constant reactor temperature of 350° C. In particular instances, the catalyst can convert 15 g, 20 g or 40 g or 50 g or even higher than 60 g of CH₃Cl per g of catalyst employed under the reaction conditions. In certain instances, the selectivity of propylene is about 50% or higher and the selectivity of butylene is about 25 to 30%. In certain instances, the C₂-C₄ olefin selectivity is greater than 85%. Further, the selectivity to C₂-C₄ alkane selectivity is less than about 1% under certain alkyl halide reaction conditions. Still further, the selectivity of aromatic compounds produced during the process is less than 1% or less than 0.1% and/or the selectivity of ethylene produced during the process is less than 10% or less than 7% or less than 5%, or less than 4%, 3%, 2%, or 1%. Still further, the selectivity of C₂-C₄ alkanes produced during the process is less than 2% or less than 0.1%.

The decrease of alkyl halide conversion can be attributed to carbon deposition on the zeolite catalyst. The carbon deposition causes the blockage of active sites resulting in decrease of conversion. The spent catalyst can be regenerated by burning of the deposited carbon. Such carbon burning can generally be performed by heating the spent catalyst under oxygen preferably diluted oxygen, often used air, at temperature between 400 to 600° C.

In another aspect of the present invention there is disclosed a zeolite catalyst capable of converting a feed that can include an alkyl halide to an olefin hydrocarbon product that include C₂ to C₄ olefins. The zeolite catalyst can include HZSM-5 having a silica to alumina (SAR) ratio of at least about 30, preferably at least about 50, and a selectivity of C₂ to C₄ olefins at least 70%, with the selectivity of propylene and butylene being at least 60%, and wherein the zeolite catalyst is capable of converting at least 40% of the alkyl halide after 20 hours of use at a temperature of 325 to 375° C. at a weight hourly space velocity (WHSV) of 0.7 to 1.0 h⁻¹. In another aspect of the present disclosure the catalyst can convert 10 g, 15 g or 20 g or 40 g or even greater than 60 g of CH₃Cl per g of catalyst under reaction conditions including temperature of about 350° C., WHSV of about 2.9 h⁻¹, reactor pressure less than 5 psig when the conversion maintained at preset level of 20% without increasing catalyst bed or reaction temperature. Similarly, the zeolite catalyst can include, in addition to or in lieu of HZSM-5, any one of or any combination of ZSM-11, ZSM-23, silicalite, ferrierite, and mordenite. Still further, the zeolite catalyst can be one that has not been treated with metal or a halide. In particular aspects, the zeolite catalyst has not been treated with a halide (e.g., it has not been subjected to fluoride treatment). In other instances, the zeolite catalyst can be treated with a metal and/or can be treated with a halide (e.g., fluoride treated).

In still another embodiment of the present invention there is disclosed a system for producing olefins. The system can include an inlet for a feed that can include the alkyl halide discussed above and throughout this specification, a reaction zone that is configured to be in fluid communication with the inlet, wherein the reaction zone includes any one of the zeolite catalysts discussed above and throughout this specification, and an outlet configured to be in fluid communication with the reaction zone to remove an olefin hydrocarbon product from the reaction zone. During use, the reaction zone can further include the alkyl halide feed and the olefin hydrocarbon product (e.g., ethylene, propylene, or butylene, or a combination thereof). The temperature of the reaction zone can be 325 to 375° C. The system can include a collection device that is capable of collecting the olefin hydrocarbon product.

In the context of the present invention, embodiments 1-26 are described. Embodiment 1 is a method for converting an alkyl halide to an olefin. The method includes contacting a zeolite catalyst that includes HZSM-5 having a silica to alumina (SAR) ratio of at least 30 with a feed that includes an alkyl halide under reaction conditions sufficient to produce an olefin hydrocarbon product that includes C₂ to C₄ olefins. The selectivity of the C₂ to C₄ olefins is at least 85% at 20% alkyl halide conversion. Embodiment 2 is the method of embodiment 1, wherein the HZSM-5 has a SAR of 30 to 150 or preferably 50 to 100. Embodiment 3 is the method of embodiments 1 and 2, wherein the HZSM-5 has bimodal acidity designated as weak acid sites and strong acid sites, wherein the HZSM-5 has a weak acid site concentration of less than 0.20 mmole/g-cat and a strong acid site concentration of greater than about 0.15 mmole/g-cat. Embodiment 4 is the method of any one of embodiments 1 to 3, wherein the reaction conditions include a temperature of greater than 300° C., a weight hourly space velocity (WHSV) of greater than 0.5 h⁻¹, and a pressure of less than 5 psig, or preferably temperature of 300 to 450° C., a weight hourly space velocity (WHSV) of 2.7 to 3.5 h⁻¹, and a pressure of less than 5 psig. Embodiment 5 is the method of any one of embodiments 1 to 4, wherein the conversion of the alkyl halide is at least about 40 g alkyl halide per g of catalyst maintaining at greater than 20% alkyl halide conversions with no change in reaction conditions. Embodiment 6 is the method of any one of embodiments 1 to 5, wherein propylene selectivity is at least 50%. Embodiment 7 is the method of any embodiments 1 to 5, wherein butylene selectivity is at least 20%. Embodiment 8 is the method of any embodiments 1 to 5, wherein aromatics selectivity less than 0.1%, and C₂-C₄ alkane selectivity is less than 2%. Embodiment 9 is the method of any one of embodiments 1 to 8, wherein the alkyl halide is a methyl halide. Embodiment 10 is the method of embodiment 9, wherein the feed includes about 10 mole % or more of a methyl halide. Embodiment 11 is the method of any one of embodiments 9 to 10, wherein the methyl halide is methyl chloride, methyl bromide, methyl fluoride, or methyl iodide, or any combination thereof. Embodiment 12 is the method of any one of embodiments 1 to 11, wherein the HZSM-5 has not been subjected to a metal treatment or halide treatment or both. Embodiment 13 is the method of any one of embodiments 1 to 12, further including collecting or storing the produced olefin hydrocarbon product. Embodiment 14 is the method of any one of embodiments 1 to 13, further including using the produced olefin hydrocarbon product to produce a petrochemical or a polymer. Embodiment 15 is the method of any one of embodiments 1 to 14, further including regenerating the used zeolite catalyst after at least 40 hours of use.

Embodiment 16 is a zeolite catalyst capable of converting a feed that includes an alkyl halide to an olefin hydrocarbon product that includes C₂ to C₄ olefins. The zeolite catalyst includes HZSM-5 having a silica to alumina (SAR) ratio of at least 30 and a selectivity of the C₂ to C₄ olefins of at least 85% at 20% alkyl halide conversion. Embodiment 17 is the zeolite catalyst of embodiment 16, wherein the HZSM-5 has a SAR of 30 to 150 or preferably 50 to 100. Embodiment 18 is the zeolite catalyst of any one of embodiments 16 to 17, wherein the HZSM-5 has bimodal acidity designated as weak acid sites and strong acid sites, wherein the HZSM-5 has a weak acid site concentration of less than 0.20 mmole/g-cat and a strong acid site concentration of greater than about 0.15 mmole/g-cat. Embodiment 19 is the zeolite catalyst of any one of embodiments 16 to 18 having a propylene selectivity of at least 50%. Embodiment 20 is the zeolite catalyst of any one of embodiments 16 to 19, having a butylene selectivity of at least 20%. Embodiment 21 is the zeolite catalyst of any one of embodiments 16 to 20, having an aromatics selectivity of less than 0.1%, and C₂-C₄ alkane selectivity of less than 2%. Embodiment 22 is the zeolite catalyst of any one of embodiments 16 to 21, wherein the HZSM-5 has not been subjected to a metal treatment or halide treatment or both.

Embodiment 23 is a system for producing olefins. The system includes an inlet for a feed that includes an alkyl halide; a reaction zone that is configured to be in fluid communication with the inlet, wherein the reaction zone includes any one of the zeolite catalysts of embodiments 16 to 22; and an outlet configured to be in fluid communication with the reaction zone to remove an olefin hydrocarbon product from the reaction zone. Embodiment 24 is the system of embodiment 23, wherein the reaction zone further includes the feed and the olefin hydrocarbon product. Embodiment 25 is the system of embodiment 24, wherein the olefin hydrocarbon product includes ethylene, propylene, and butylene. Embodiment 26 is the system of any one of embodiments 23 to 25, further including a collection device that is capable of collecting the olefin hydrocarbon product.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, basic and novel characteristics of the catalysts of the present invention are their ability to selectivity produce an olefin, and in particular, propylene and butylene, in high amounts, while also remaining stable/activated after prolonged periods of use (e.g., 20 hours).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1: Chart listing various chemicals and products that can be produced from propylene.

FIG. 2: Schematic of an embodiment of a system for producing olefins from alkyl halides.

FIG. 3: NH₃-TPD of HZSM-5 catalysts of the present invention. Curve identified by letter references the related catalyst in the Examples (e.g., curve A refers Catalyst A).

FIG. 4: Acidity of HZSM-5 catalysts of the present invention (measured by NH₃-TPD) as a function of SiO₂/Al₂O₃ ratio. Curves 1, 2 and 3 represent weak, strong and total acid sites, respectively.

FIG. 5: Conversion of methyl chloride reaction over HZSM-5 catalysts of the present invention as function of time-on-stream (refer Table 3 for reaction conditions). Curves A through H refer Catalysts A through H, respectively.

FIG. 6: Product selectivity of methyl chloride reaction at 20 h over HZSM-5 catalysts (refer Table 3 for reaction conditions) of the present invention as a function of silica to alumina ratio (SAR).

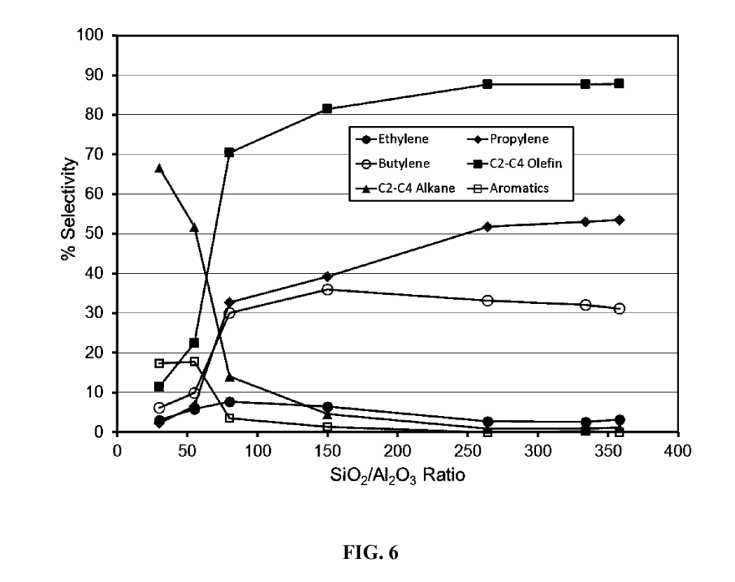

FIG. 7: CH₃Cl conversions as a function of time-on-stream over various HZSM-5 catalyst under conditions: temperature about 350° C., WHSV about 2.8 h⁻¹, pressure of about 2 psig (refer Table 5 for reaction conditions).

FIG. 8: CH₃Cl conversion (g per g of catalyst) at conditions of Table 5 until the conversions reached to 20% at the constant temperature as a function of SAR of HZSM-5.

### DETAILED DESCRIPTION OF THE INVENTION

In the petrochemical industry, the principal source for light olefins (ethylene and propylene) is steam cracking of hydrocarbons, for example, naphtha, LPG or ethane. Using alternative feedstock such as methane has been an attractive alternative by converting the methane to light olefins via a two-step process (conversion of methane to methyl halide particularly to methyl mono-halide, for example, to methyl chloride followed by conversion of the halide to C₂-C₄ olefins). Unfortunately, however, this alternative has been largely unsuccessful. For instance, zeolite (e.g., ZSM-5) and zeolite type catalysts (e.g., SAPO-34) have been tried for the methyl chloride conversion, but the selectivity to a desired olefin (e.g., propylene) and the rapid catalyst deactivation for the halide reaction remains the major challenge to such a process.

A discovery has been made with ZSM-5 zeolite catalysts that alleviate such problems. In particular, the use of untreated HZSM-5 zeolite catalysts having a high silica to alumina ratio (SAR) of at least about 30, but less than 1000, surprisingly results in an increase in propylene and butylene production selectivity from alkyl halides. The HZSM-5 with a particular SAR substantially improve catalyst deactivation giving an increased CH₃Cl conversion under methyl halide reaction conditions with greater than 85% C₂-C₄ olefin selectivity. This allows for a more targeted and continuous production of the olefins without having to constantly provide additional catalyst to the reaction process.

These and other non-limiting aspects of the present invention are discussed in further detail in the following sections.

### A. ZSM-5 Catalysts

ZSM-5 zeolite is a porous material containing intersecting two-dimensional pore structure with 10-membered ring openings. This zeolite and its preparation are described in U.S. Pat. No. 3,702,886, which is herein incorporated by reference. In the present invention, the ZSM-5 zeolite may include those having a silica to alumina (SiO₂/Al₂O₃) ratio of at least about 30, but less than 1000. Additionally, modified and unmodified ZSM-5 zeolites are commercially available from a wide range of sources (e.g., Zeolyst International, Valley Forge, Pa., USA, Clariant International Ltd., Munich, Germany, Tricat Inc., McAlester, Okla., USA). In preferred embodiments, unmodified HZSM-5 is used, which is commercially available from at least the aforementioned sources. However, modified ZSM-5 as well as other zeolites such as ZSM-11, ZSM-23, silicalite, ferrierite, and mordenite can also be used. While the SAR for each of the zeolites can vary, in preferred aspects, a SAR of at least 30 for the additional zeolites is preferred.

In certain aspects, the ZSM-5 zeolite catalyst of the present invention is acidic or H-form and can be synthesized or commercially obtained from sources. Several NH₄-form ZSM-5 zeolite powder samples were obtained from Zeolyst International Inc. and used in the Examples. The SARs of as-received zeolites are 30, 55, 80, 150, 264, 334, 358 and 1192. The NH₄-form zeolites were calcined in air at 530° C. for 10 h and were then used as catalysts for methyl chloride conversion reaction.

The HZSM-5 catalysts exhibit bimodal acidity and have two major broad peaks as characterized by ammonia temperature programmed desorption (NH₃-TPD) technique, one with peak maximum between 150° C. and 200° C., and the other with peak maximum between 250° C. and 400° C. The lower temperature peak is attributed to weak acid sites while the higher temperature peak is attributed to strong acid sites. In a preferred aspect of the present invention, the HZSM-5 catalyst can have weak acid sites or acidity of less than about 0.20 mmole/g-cat. The HZSM-5 catalyst can also have strong acid sites or acidity of greater than about 0.15 mmole/g-cat or preferably greater than about 0.20 mmole/g-cat.

### B. Alkyl Halide Feed

The alkyl halide feed includes one or more alkyl halides. The alkyl halide feed may contain alkyl mono halides, alkyl dihalides, alkyl trihalides, preferably alkyl mono halide with less than 10% of other halides relative to the total halides. The alkyl halide feed may also contain nitrogen, helium, steam, and so on as inert compounds. The alkyl halide in the feed may have the following structure: C₁H₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. Non-limiting examples of alkyl halides include methyl chloride, methyl bromide, methyl fluoride, or methyl iodide, or any combination thereof. In particular aspects, the feed may include about 10, 15, 20, 40, 50 mole % or more of the alkyl halide. In particular embodiments, the feed contains up to 20 mole % of the feed includes an alkyl halide. In preferred aspects, the alkyl halide is methyl chloride. In a particular embodiment, the alkyl halide is methyl chloride or methyl bromide.

The production of alkyl halide, particularly of methyl chloride (CH₃Cl, See Equation 1 below), is commercially produced by thermal chlorination of methane at 400° C. to 450° C. and at a raised pressure. Catalytic oxychlorination of methane to methyl chloride is also known. In addition, methyl chloride is industrially made by reaction of methanol and HCl at 180° C. to 200° C. using a catalyst. Alternatively, methyl halides are commercially available from a wide range of sources (e.g., Praxair, Danbury, Conn.; Sigma-Aldrich Co. LLC, St. Louis, Mo.; BOC Sciences USA, Shirley, N.Y.). In preferred aspects, methyl chloride and methyl bromide can be used alone or in combination.

### C. Olefin Production

The HZSM-5 catalysts of the present invention help to catalyze the conversion of alkyl halides to C₂-C₄ olefins such as ethylene, propylene and butylenes. The following non-limiting two-step process is an example of conversion of methane to methyl chloride and conversion of methyl chloride to ethylene, propylene and butylene. The second step illustrates the reactions that are believed to occur in the context of the present invention:

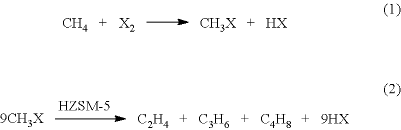

where X is Br, F, I, or Cl. Besides the C₂-C₄ olefins the reaction may produce byproducts such as methane, C₅ olefins, C₂-C₅ alkanes and aromatic compounds such as benzene, toluene and xylene.

Conditions sufficient for olefin production (e.g., ethylene, propylene and butylene as shown in Equation 2) include temperature, time, alkyl halide concentration, space velocity, and pressure. The temperature range for olefin production may range from about 300° C. to 500° C., preferably ranging 350° C. to 450° C. In more preferred aspects, the temperature range is from 325° C. to 375° C. A weight hourly space velocity (WHSV) of alkyl halide higher than 0.5 h⁻¹ can be used, preferably between 0.5 and 10 h⁻¹, more preferably between 0.5 and 5 h⁻¹, even more preferably between 0.5 to 3 h⁻¹. The conversion of alkyl halide is carried out at a pressure less than 200 psig preferably less than 100 psig, more preferably less than 50 psig, even more preferably less than 20 psig. The conditions for olefin production may be varied based on the type of the reactor.

The reaction can be carried out over the HZSM-5 having the particular SAR for prolonged periods of time without changing or re-supplying new catalyst or catalyst regeneration. This is due to the stability or slower deactivation of the catalysts of the present invention. Therefore, the reaction can be performed for a period until the level of alkyl halide conversion reaches to a preset level (e.g., 20%). In preferred aspects, the reaction is continuously run for 20 h or 20 h to 50 h or longer without having to stop the reaction to resupply new catalyst or catalyst regeneration. The method can further include collecting or storing the produced olefin hydrocarbon product along with using the produced olefin hydrocarbon product to produce a petrochemical or a polymer.

### D. Catalyst Activity/Selectivity

Catalytic activity as measured by alkyl halide conversion can be expressed as the % moles of the alkyl halide converted with respect to the moles of alkyl halide fed. In particular aspects, the combined selectivity of ethylene, propylene and butylene is at least 85% under certain reaction conditions. In certain instances, the selectivity of propylene is about 50% or higher, the selectivity of butylene is about 25% or higher, and ethylene selectivity is about 5% or less. Still further, the selectivity of aromatic compounds produced during the process is less than 1% or less than 0.5% and the selectivity of C₂-C₄ alkanes is less than about 2% or less than 1% under certain reaction conditions. As an example, methyl chloride (CH₃Cl) is used here to define conversion and selectivity of products by the following formulas:

$$\% {CH}_{3} Cl Conversion = \frac{( {CH}_{3} Cl ) ° - ( {CH}_{3} Cl )}{( {CH}_{3} Cl ) °} \times 100$$

where, (CH₃Cl)° and (CH₃Cl) are moles of methyl chloride in the feed and reaction product, respectively.

Selectivity is defined as C-mole % and are defined for ethylene, propylene, and so on as follows:

$$\% Ethylene Selectivity \times 100 = \frac{2 ( {C}_{2} {H}_{4} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) +\end{matrix}}$$

where, the numerator is the carbon adjusted mole of ethylene and the denominator is the sum of all the carbon adjusted mole of all hydrocarbons in the product stream.

Selectivity for propylene may be expressed as:

$$\% Propylene Selectivity \times 100 = \frac{3 ( {C}_{3} {H}_{6} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) + …\end{matrix}}$$

where, the numerator is the carbon adjusted mole of propylene and the denominator is the sum of all the carbon adjusted mole of all hydrocarbons in the product stream.

Selectivity for butylene may be expressed as:

$$\% Butylene Selectivity \times 100 = \frac{4 ( {C}_{4} {H}_{8} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) + …\end{matrix}}$$

where, the numerator is the carbon adjusted mole of butylene and the denominator is the sum of all the carbon adjusted mole of all hydrocarbons in the product stream.

Selectivity for aromatic compounds may be expressed as:

$$\% Aromatics Selectivity \times 100 = \frac{6 ( {C}_{6} {H}_{6} ) + 7 ( {C}_{7} {H}_{8} ) + 8 ( {C}_{8} {H}_{10} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) + …\end{matrix}}$$

where, the numerator is the carbon adjusted moles of aromatics (benzene, toluene and xylene) and the denominator is the sum of all the carbon adjusted mole of all hydrocarbons in the product stream.

### E. Olefin Production System

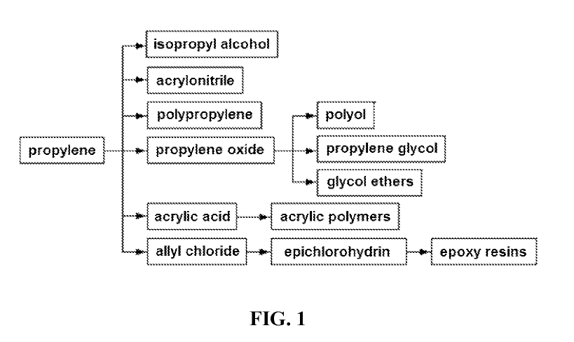

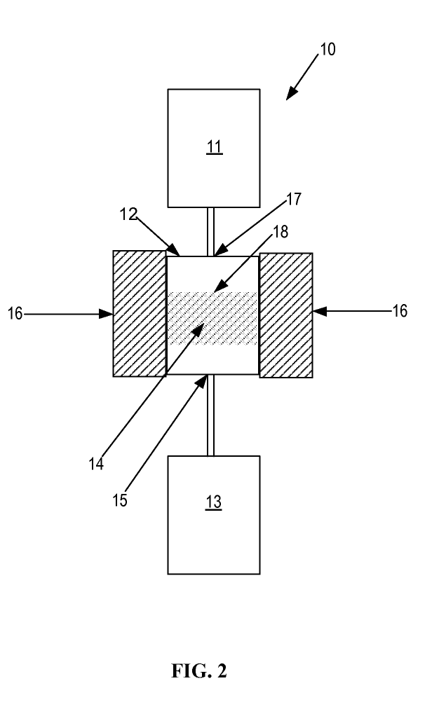

Referring to FIG. 2, a system 10 is illustrated, which can be used to convert alkyl halides to olefin hydrocarbon products with the ZSM-5 zeolite catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the ZSM-5 zeolite catalyst 14 of the present invention. The amounts of the alkyl halide feed 11 and the catalyst 14 used can be modified as desired to achieve a given amount of product produced by the system 10. Non-limiting examples of reactors that can be used include fixed-bed reactors, fluidized bed reactors, bubbling bed reactors, slurry reactors, rotating kiln reactors, or any combinations thereof when two or more reactors are used. In preferred aspects, reactor 12 that can be used is a fixed-bed reactor (e.g., a fixed-bed tubular quartz reactor which can be operated at atmospheric pressure). The reactor 12 can include an outlet 15 for products produced in the reaction zone 18. The products produced can include ethylene, propylene and butylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., C₂-C₄ olefins) for other uses. By way of example only, FIG. 1 provides non-limiting uses of propylene produced from the catalysts and processes of the present invention. Still further, the system 10 can also include a heating source 16. The heating source 16 can be configured to heat the reaction zone 18 to a temperature sufficient (e.g., 325 to 375° C.) to convert the alkyl halides in the alkyl halide feed to olefin hydrocarbon products. A non-limiting example of a heating source 16 can be a temperature controlled furnace. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Catalyst Preparation

NH₄-ZSM-5 (NH₄-form ZSM-5) zeolite powder samples were obtained from Zeolyst International Inc. The NH₄-form zeolites were calcined in air at 530° C. for 10 h to transform them into H-form (HZSM-5) zeolites, and were then used as catalysts for methyl chloride conversion reaction. HZSM-5 zeolites (designated as Catalysts A through H) with their SAR are shown in Table 2.

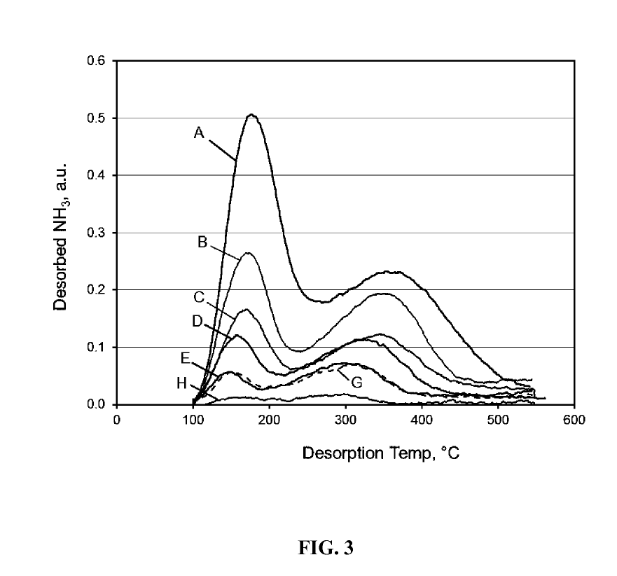

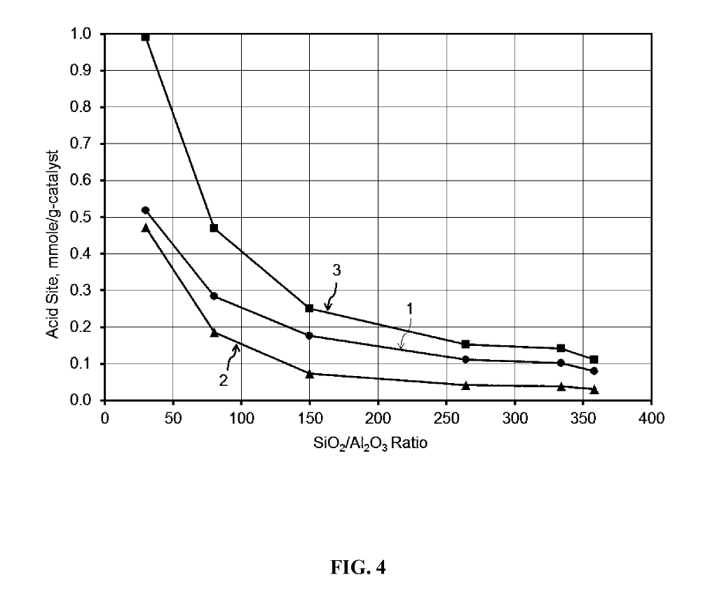

The acidity of each of the HZSM-5 catalysts was measured by NH₃-TPD. Generally, a temperature at which NH₃ being desorbed is an estimation of strength of acid site, e.g., higher the desorption temperature stronger is the acid site. FIG. 3 shows NH₃-TPD of various HZSM-5 catalysts. All the ZSM-5 catalysts used in this disclosure show two NH₃-TPD peaks. Acidity or acid site density (mmole/g-catalyst) was measured from the amount NH₃ desorbed under the peak and the results are listed in Table 2. FIG. 4 shows acid site as a function of SAR of ZSM-5 catalysts. Catalyst A having SAR 30 shows one peak with peak maximum around 175° C. and the other peak maximum around 356° C. As the SAR of the HZSM-5 increases the peak maxima for both peaks continuously shifted to lower temperature. Also, the amount of acid sites (mmole/g-catalyst) or acid (site) density decreases gradually with the increase of SAR of the zeolite.

TABLE 2

| | | Acidity,^{1,2} mmole/g-catalyst | | |
|---|---|---|---|---|
| Catalyst | SAR | Weak | Strong | Total |
| A | 30 | 0.47 (175° C.) | 0.52 (356° C.) | 0.99 |
| B | 55 | 0.19 (167° C.) | 0.28 (344° C.) | 0.47 |
| C | 80 | 0.12 (166° C.) | 0.21 (339° C.) | 0.33 |
| D | 150 | 0.07 (155° C.) | 0.18 (322° C.) | 0.25 |
| E | 264 | 0.04 (148° C.) | 0.11 (300° C.) | 0.15 |
| F | 334 | 0.04 (144° C.) | 0.10 (307° C.) | 0.14 |
| G | 358 | 0.03 (151° C.) | 0.08 (305° C.) | 0.11 |
| H | 1192 | 0.02 (150° C.) | 0.03 (284° C.) | 0.05 |

¹Acidity of HZSM-5 zeolite catalysts was measured by NH₃-TPD using Micromeritics Autochem II 2920 Analyzer. A measured amount of powder catalyst (typically 0.2 g) was placed in a sample tube, heated under He flow (30 sccm) at 550° C. for 2 h, cooled to 100° C. at which time helium flow was replaced by NH₃ flow (50 sccm, 5 mole % NH₃ in He) for 1 h, and then NH₃ flow was replaced by He flow. Once the base line was established, the NH₃ was desorbed by heating at 10° C./min to 550° C. under He flow.
²Peak maxima of NH₃-TPD peaks are given in parentheses.

### Examples 1-8

### Methyl Chloride Conversion to Olefins at about 350° C., WHSV 0.9 h¹ and <5 psig

Each of the powder catalysts A through H were first pressed into tablet and then crushed and sieved between 20 and 40 mesh screens. A measured amount of the 20-40 mesh sized catalysts (typically 3.0 g) were loaded in a tubular (SS-316, ½-inch OD) reactor. The catalyst was dried under N₂ flow (100 cm³/min) at 200° C. for 1 h and then raised to 300° C. when N₂ flow was replaced by methyl chloride (CH₃Cl) (20 mole %, balance N₂) (flow rate 90 cm³/min). The weight hourly space velocity (WHSV) of CH₃Cl was about 0.9 h¹. The reactor inlet pressure was 2.2 to 2.8 psig. After an initial period of reaction at 300° C. for about 2 to 3 h the catalyst bed temperature was raised to about 350° C. Catalyst loading, feed rate, space velocity, catalyst bed temperature, and reactor (inlet) pressure for examples 1-8 are summarized in Table 3. The methyl chloride feed and product stream were analyzed to determine conversion and product selectivity as described earlier.

TABLE 3

| | | | | | | Reactor |
|---|---|---|---|---|---|---|
| | | | | | Catalyst | Inlet |
| Ex- | | Catalyst | Feed Rate¹ | WHSV | Bed Temp | Pressure |
| ample | Catalyst | (g) | (cm3/min) | (h⁻¹) | (° C.) | (psig) |
| 1 | A | 3.01 | 90 | 0.9 | 348 | 2.7 |
| 2 | B | 3.01 | 90 | 0.9 | 347 | 2.7 |
| 3 | C | 3.00 | 90 | 0.9 | 350 | 2.8 |
| 4 | D | 3.02 | 90 | 0.9 | 348 | 2.7 |
| 5 | E | 3.00 | 90 | 0.9 | 348 | 2.8 |
| 6 | F | 3.01 | 90 | 0.9 | 349 | 2.8 |
| 7 | G | 3.01 | 90 | 0.9 | 349 | 2.2 |
| 8 | H | 3.01 | 90 | 0.9 | 350 | 2.8 |

¹Total feed rate (feed contains 20 mole % CH₃Cl in N₂)

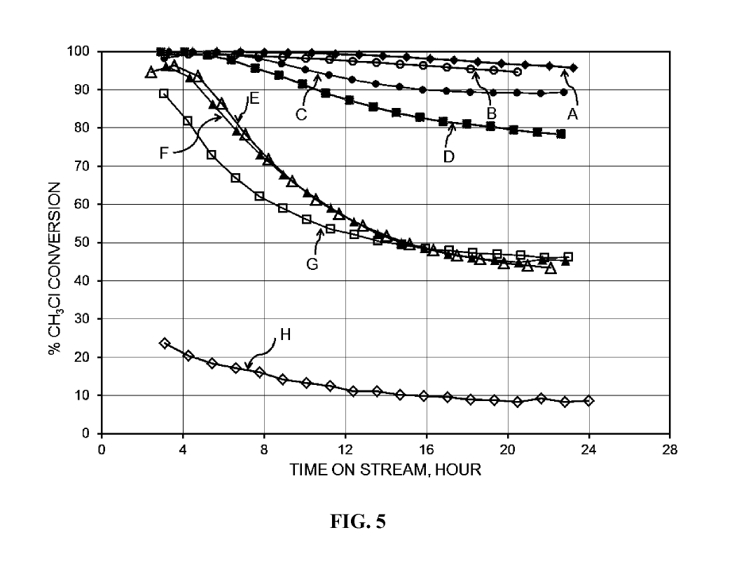

FIG. 5 shows CH₃Cl conversion over the ZSM-5 catalysts as a function of time on stream. Also, CH₃Cl conversion and product selectivity for the HZSM-5 catalysts at 20 h run time are shown in Table 4. Under the reaction conditions used all the catalysts show gradual decrease in CH₃Cl conversion with time on stream. Catalysts A and B show greater than 90% CH₃Cl conversion at 20 h with less than 25% C₂-C₄ olefins selectivity, greater than 50% C₂-C₄ alkane selectivity and greater than 15% aromatics selectivity. Whereas Catalysts C and D show about 80-90% CH₃Cl conversion at 20 h with greater than about 70% C₂-C₄ olefins selectivity, less than 15% C₂-C₄ paraffin selectivity and less than 5% aromatics selectivity. Catalysts E, F and G show about 45% CH₃Cl conversion at 20 h with greater about 85% C₂-C₄ olefins selectivity, less than 1% C₂-C₄ paraffin selectivity and less than 0.5% aromatics selectivity. Catalyst H shows about 9% conversion. Catalysts C through H showed about 70% or higher C₂-C₄ olefins with combined propylene and butylene selectivity of about 60%.

TABLE 4

| | | | | % Selectivity² | | | | | | |
|---|---|---|---|---|---|---|---|---|---|---|
| | | | % CH₃Cl | C₂ | C₃ | C₄ | C₂-C₄ | C₂-C₄ | | |
| Example | Catalyst¹ | | Conv.² | olefin | olefin | olefin | olefin | alkane | C₅₊ | Aromatics |
| 1 | A | (30) | 96.6 | 3.0 | 2.2 | 6.2 | 11.4 | 66.7 | 2.4 | 17.3 |
| 2 | B | (55) | 94.9 | 5.8 | 6.7 | 9.9 | 22.4 | 51.8 | 4.9 | 17.8 |
| 3 | C | (80) | 89.2 | 7.7 | 32.7 | 30.0 | 70.4 | 14.1 | 8.3 | 3.5 |
| 4 | D | (150) | 79.9 | 6.4 | 39.2 | 35.9 | 81.5 | 4.6 | 6.6 | 1.3 |
| 5 | E | (264) | 44.6 | 2.7 | 51.8 | 33.2 | 87.7 | 0.9 | 8.2 | 0 |
| 6 | F | (334) | 45.1 | 2.6 | 53.0 | 32.1 | 87.7 | 0.9 | 6.5 | 0.2 |
| 7 | G | (358) | 46.9 | 3.2 | 53.5 | 31.1 | 87.8 | 1.2 | 6.6 | 0 |
| 8 | H | (1192) | 8.6 | 6.5 | 60.2 | 21.7 | 88.4 | 0 | 7.4 | 0 |

¹Number in parenthesis is the silica to alumina ratio (SAR).
²Reaction conditions: Catalyst 3.0 g, feed 20% CH₃Cl (balance N₂), WHSV 0.9 h⁻¹, feed introduced at 300° C. and then raised to 350° C. after about 2 h. Conversion and selectivity were calculated at 20 h time on stream.

### Examples 9-16

### Methyl Chloride Conversion to Olefins at about 350° C., WHSV 2.75-2.91 h¹ and <5 psig

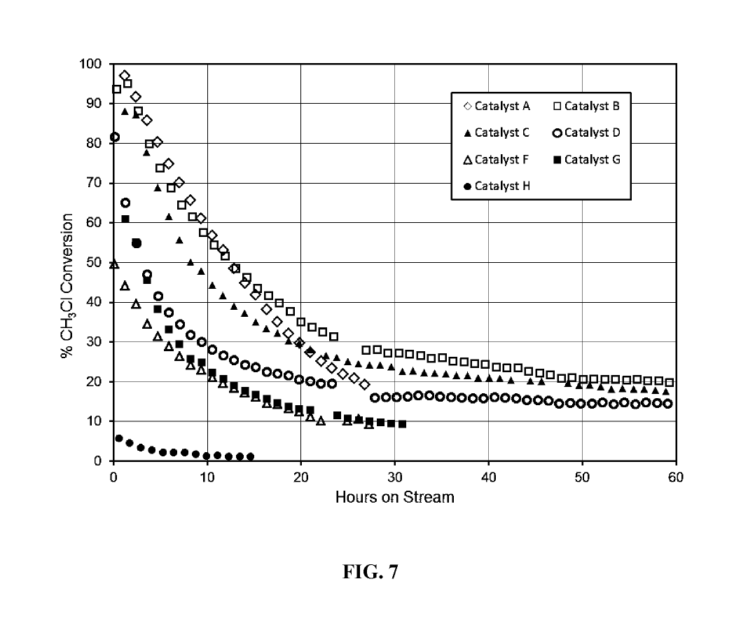

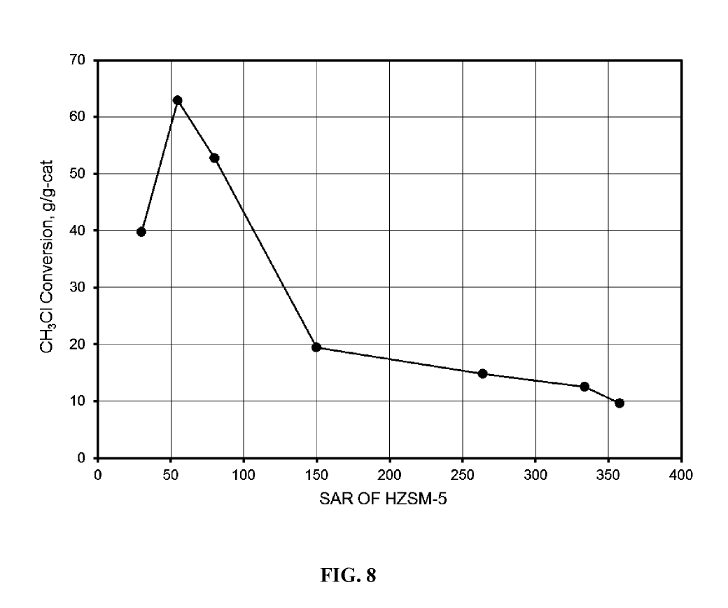

Each of Catalysts A-H (typically 1.0 g, sized 20-40 mesh) was loaded in a reactor. The catalyst was dried under N₂ flow (100 cm³/min) at 200° C. for 1 h and then raised to 350° C. when N₂ flow was replaced by methyl chloride (CH₃Cl) (20 mole %, balance N₂) (flow rate 100 cm³/min). The weight hourly space velocity (WHSV) of CH₃Cl was about 2.75 to 2.91 h⁻¹. The reactor inlet pressure was 1.6 to 2.5 psig. Catalyst load, feed rate, space velocity, catalyst bed temperature, and reactor (inlet) pressure for examples 9-16 are summarized in Table 5. The methyl chloride feed and product stream were analyzed to determine conversion and product selectivity as described earlier. FIG. 7 shows CH₃Cl conversion as a function of time on stream over the ZSM-5 catalysts. All the catalysts show decrease in conversion with time on stream, with a sharp decrease in conversion being observed during initial periods and then the conversion decreases gradually. The catalyst test continued until the conversion decreased to 20% (or lower) maintaining the same test conditions. The catalyst conversion (g of CH₃Cl converted per g of catalyst until gradually reached to 20% conversion at 350° C.), initial conversion and selectivity, and selectivity at 20% conversion are summarized in Table 6. FIG. 8 shows CH₃Cl conversion as a function of SAR of HZSM-5 catalysts used in the examples. Catalyst H showed lower than 20% CH₃Cl conversion at the conditions used and therefore its conversion capacity was not calculated.

TABLE 5

| | | | | | | Reactor |
|---|---|---|---|---|---|---|
| | | | | | | Inlet |
| Ex- | | Catalyst | Feed Rate¹ | WHSV | Bed Temp | Pressure |
| ample | Catalyst | (g) | (cm3/min) | (h⁻¹) | (° C.) | (psig) |
| 9 | A | 1.01 | 100 | 2.9 | 348 | 2.5 |
| 10 | B | 1.00 | 100 | 2.9 | 349 | 2.5 |
| 11 | C | 1.00 | 100 | 2.8 | 349 | 1.6 |
| 12 | D | 1.01 | 100 | 2.8 | 349 | 1.6 |
| 13 | E | 1.01 | 100 | 2.7 | 349 | 1.6 |
| 14 | F | 1.01 | 100 | 2.8 | 351 | 1.6 |
| 15 | G | 1.01 | 100 | 2.8 | 351 | 1.8 |
| 16 | H | 1.01 | 100 | 2.8 | 343 | 1.6 |

¹Total feed rate (feed contains 20 mole % CH₃Cl in N₂)

TABLE 6

| | | | CH₃Cl Conversion² | | % Initial Selectivity³ | | | | | | % Selectivity at 20% Conv. | | | | | |
|---|---|---|---|---|---|---|---|---|---|---|---|---|---|---|---|---|
| Ex- | | | % Initial | gCH₃Cl/ | C₂ | C₃ | C₄ | C₂-C₄ | C₂-C₄ | Aro- | C₂ | C₃ | C₄ | C₂-C₄ | C₂-C₄ | Aro- |
| ample | Catalyst¹ | | Conv. | g-cat | olefin | olefin | Olefin | Olefin | alkane | matics | olefin | olefin | olefin | Olefin | alkane | matics |
| 9 | A | (30) | 100.0 | 39.8 | 4.3 | 3.3 | 7.8 | 15.4 | 62.8 | 14.9 | 7.5 | 48.7 | 30.4 | 86.6 | 5.6 | 0 |
| 10 | B | (55) | 95.8 | 62.8 | 8.2 | 10.1 | 12.1 | 30.4 | 45.3 | 14.4 | 7.0 | 55.2 | 24.4 | 86.6 | 1.4 | 0 |
| 11 | C | (80) | 95.8 | 52.7 | 11.2 | 20.1 | 21.7 | 53.0 | 27.6 | 4.9 | 3.9 | 58.7 | 26.1 | 88.7 | 0 | 0 |
| 12 | D | (150) | 82.1 | 19.4 | 8.1 | 31.1 | 29.6 | 68.8 | 10.5 | 2.3 | 1.6 | 59.5 | 27.8 | 88.9 | 0 | 0 |
| 13 | E | (264) | 73.1 | 14.8 | 7.2 | 41.4 | 22.3 | 70.9 | 7.9 | 0.7 | 1.9 | 56.4 | 28.2 | 86.5 | 0 | 0 |
| 14 | F | (334) | 61.1 | 12.5 | 5.7 | 42.7 | 23.0 | 71.4 | 10.4 | 0.4 | 1.9 | 57.2 | 27.2 | 86.3 | 0 | 0 |
| 15 | G | (358) | 49.5 | 9.7 | 5.7 | 47.0 | 24.4 | 77.1 | 7.7 | 0.4 | 5.8 | 59.4 | 27.3 | 92.5 | 0 | 0 |
| 16 | H | (1192) | 5.7 | | 4.3 | 60.8 | 23.0 | 88.1 | 0 | 0 | | | | | | |

¹Number in parenthesis is the silica to alumina ratio (SAR).
²Initial conversion estimated at 0.1 h reaction time from conversion vs. time-on-stream plot. CH₃Cl conversion gCH₃Cl/g-catalyst was calculated until the conversion reached to 20% at constant temperature of about 350° C.
³Selectivity was obtained at reaction time at about 0.1 h.

1. A method for converting an alkyl halide to an olefin, the method comprising contacting a zeolite catalyst comprising HZSM-5 having a silica to alumina (SAR) ratio of at least 30 with a feed comprising an alkyl halide under reaction conditions sufficient to produce an olefin hydrocarbon product comprising C₂ to C₄ olefins, wherein the selectivity of the C₂ to C₄ olefins is at least 85% at 20% alkyl halide conversion.

2. The method of claim 1, wherein the HZSM-5 has a SAR of 30 to 150 or 50 to 100.

3. The method of claim 1, wherein the HZSM-5 has bimodal acidity designated as weak acid sites and strong acid sites, wherein the HZSM-5 has a weak acid site concentration of less than 0.20 mmole/g-cat and a strong acid site concentration of greater than about 0.15 mmole/g-cat.

4. The method of claim 1, wherein the reaction conditions include a temperature of greater than 300° C., a weight hourly space velocity (WHSV) of greater than 0.5 h⁻¹, and a pressure of less than 5 psig, or preferably temperature of 300 to 450° C., a weight hourly space velocity (WHSV) of 2.7 to 3.5 h⁻¹, and a pressure of less than 5 psig.

5. The method of claim 1, wherein the conversion of the alkyl halide is at least about 40 g alkyl halide per g of catalyst maintaining at greater than 20% alkyl halide conversions with no change in reaction conditions.

6. The method of claim 1, wherein propylene selectivity is at least 50%.

7. The method of claim 1, wherein butylene selectivity is at least 20%.

8. The method of claim 1, wherein aromatics selectivity less than 0.1%, and C₂-C₄ alkane selectivity is less than 2%.

9. The method of claim 1, wherein the alkyl halide is a methyl halide.

10. The method of claim 9, wherein the feed comprises about 10 mole % or more of a methyl halide.

11. The method of claim 1, wherein the HZSM-5 has not been subjected to a metal treatment or halide treatment or both.

12. The method of claim 1, further comprising collecting or storing the produced olefin hydrocarbon product.

13. The method of claim 1, further comprising using the produced olefin hydrocarbon product to produce a petrochemical or a polymer.

14. The method of claim 1, further comprising regenerating the used zeolite catalyst after at least 40 hours of use.

15. A zeolite catalyst capable of converting a feed comprising an alkyl halide to an olefin hydrocarbon product comprising C₂ to C₄ olefins, the zeolite catalyst comprising HZSM-5 having a silica to alumina (SAR) ratio of at least 30 and a selectivity of the C₂ to C₄ olefins of at least 85% at 20% alkyl halide conversion.

16. The zeolite catalyst of claim 15, wherein the HZSM-5 has a SAR of 30 to 150 or preferably 50 to 100.

17. The zeolite catalyst of claim 15, wherein the HZSM-5 has bimodal acidity designated as weak acid sites and strong acid sites, wherein the HZSM-5 has a weak acid site concentration of less than 0.20 mmole/g-cat and a strong acid site concentration of greater than about 0.15 mmole/g-cat.

18. The zeolite catalyst of claim 15, wherein the catalyst has a propylene selectivity of at least 50%, a butylene selectivity of at least 20%, an aromatics selectivity of less than 0.1%, and C₂-C₄ alkane selectivity of less than 2%, or any combination thereof.

19. The zeolite catalyst of claim 15, wherein the HZSM-5 has not been subjected to a metal treatment or halide treatment or both.

20. A system for producing olefins, the system comprising:
an inlet for a feed comprising an alkyl halide;
a reaction zone that is configured to be in fluid communication with the inlet, wherein the reaction zone comprises the zeolite catalyst of claim 15; and
an outlet configured to be in fluid communication with the reaction zone to remove an olefin hydrocarbon product from the reaction zone.

21. The system of claim 20, wherein the olefin hydrocarbon product comprises ethylene, propylene, and butylene.
